(19) United States
(12) Patent Application Publication
(10) Pub. No.: US 2007/0281927 A1
(43) Pub. Date: Dec. 6, 2007

(54) Anti-inflammatory and analgesic compositions and related methods

(76) Inventors: Shanthakumar Tyavanagimatt, Salt Lake City, UT (US); David Fikstad, Salt Lake City, UT (US); Chandrashekar Giliyar, Salt Lake City, UT (US); Mahesh Patel, Salt Lake City, UT (US); Srinivasan Venkateshwaran, Salt Lake City, UT (US)

Correspondence Address: THORPE NORTH & WESTERN, LLP., 8180 SOUTH 700 EAST, SUITE 350, SANDY, UT, 84070, US

(21) Appl. No.: 11/448,597

(22) Filed: Jun. 6, 2006

(51) Int. Cl. A61K 31/5415

(52) U.S. Cl. 514/226.5

(57) ABSTRACT

Methods and compositions for delivering a meloxicam compound are disclosed and described. In one aspect, a method may include perorally administering to a subject a therapeutically effective amount of a meloxicam compound that provides a meloxicam plasma concentration within 1 hour which is at least about 40% of the maximum plasma concentration attained by the formulation. In another aspect, a composition may include a therapeutically effective amount of a meloxicam compound in a pharmaceutically acceptable carrier including at least one of an alkalizer or a solubilizer, with the meloxicam compound having a solubility in the carrier that is greater than about 1.0 mg/gm.

### FIELD OF THE INVENTION

The present invention relates to oral dosage formulations and methods for providing rapidly absorbed non-steroidal anti-inflammatory agents to a subject for the treatment or prevention of various medical conditions. Accordingly, this invention involves the fields of chemistry, pharmaceutical sciences, medicine and other health sciences.

### BACKGROUND OF THE INVENTION

Non-steroidal anti-inflammatory drugs (NSAIDs) are useful in treating acute and chronic pain, as well as inflammation. It is believed that one major mechanism of action of many NSAIDs is the inhibition of the cyclooxygenase (COX) enzyme system, resulting in decreased prostaglandin synthesis. As such, these compounds may be particularly useful to provide adequate pain management for many individuals without producing many of the side effects and dependencies prevalent with opioid pain management.

Meloxicam is an example of an NSAID with anti-inflammatory, antipyretic, and analgesic activity. Meloxicam is an NSAID that is poorly soluble at the acidic pH range of the upper gastrointestinal tract and is thus absorbed slowly with a time delay after oral administration. The time to maximum plasma concentration of meloxicam for many individuals is typically achieved 5 hours or more after oral administration of the drug. Meloxicam is currently marketed under the name MOBIC® by Boehringer Ingelheim Pharmaceuticals, Inc., provided in 7.5 mg and 15 mg tablet dosage form. The bioavailability of a single 30 mg oral dose is 89% as compared to a 30 mg intravenous bolus injection. Under fasted, steady-state conditions, the mean maximum plasma concentration of meloxicam (Cₘₐₓ) is achieved within four to five hours, with a second meloxicam concentration peak occurring at approximately twelve to fourteen hours post-dose, which suggests gastrointestinal recirculation.

MOBIC® is approved by the FDA for the relief of the signs and symptoms of osteoarthritis and rheumatoid arthritis and the recommended starting and maintenance oral dose of MOBICS is 7.5 mg to 15 mg once daily. Meloxicam is known for the treatment of many indications, including without limitation acute pain and chronic pain from a wide variety of sources (nociceptive and neuropathic); osteoarthritis; rheumatoid arthritis; juvenile polyarticular arthritis; ankylosing spondylitis; migraine; amyotrophic lateral sclerosis; diabetes related ocular disorders; cardiovascular disorders, including acute coronary syndromes; polycystic kidney disease; cancer; preterm labor; prostatitis or pelvic pain syndrome; organ injury during transplantation; psychiatric disorders including schizophrenia, delusional disorders, affective disorders, autism or tic disorders; obesity; urinary incontinence; and immunodeficiency.

Examples of such use may be found in the MOBIC® prescribing information from Boehringer Ingelheim GmbH, Germany, 2006; US Application No. 2004063752; PCT Application No. WO2005/049014; US Application No. 2002/035156; Circulation 106:191-195 (2002); US Application No. 2004/024042; Critical Reviews in Clinical Laboratory Sciences 37(5):431-502 (2002); PCT Application WO97/31631; U.S. Pat. No. 6,403,640; US Patent Application No. 2004/204469; US Patent Application No. 2004/204472; U.S. Pat. No. 6,440,963; and US Patent Application No. US2004/082640, each of which is incorporated herein by reference.

Because of the problems associated with acute and chronic pain and management of pain with opioids, there is a considerable need for a rapidly absorbed meloxicam composition as a means to decrease the time to maximum plasma concentration and time to onset of action of meloxicam while maintaining the benefits of once daily administration.

In view of the foregoing, compositions and methods for administering meloxicam having a more rapid onset of analgesic and anti-inflammatory activity continue to be sought.

### SUMMARY OF THE INVENTION

Accordingly, the present invention provides rapidly absorbing meloxicam compositions that reduce the time to reach effective plasma concentrations of meloxicam, and thus may decrease the time to onset of analgesic and/or anti-inflammatory efficacy for orally administered meloxicam. In one aspect of the present invention, methods of providing meloxicam therapy to a subject are provided. One example of such a method may include perorally administering to the subject a therapeutically effective amount of a meloxicam compound from a composition that provides a meloxicam plasma concentration during the period from about 0 to 1 hours after administration which is at least about 40% of the maximum plasma concentration or Cmax attained by the formulation. In some aspects, such a plasma concentration may be achieved within about 20 minutes of administering of the composition to a subject. In another aspect, the meloxicam plasma concentration during a period from about 0 to 1 hours after administration is at least about 80% of the maximum plasma concentration attained by the formulation. In some aspects, the maximum plasma concentration may be the maximum concentration observed between about 2 hours and about 10 hours after administration of the composition. In yet another aspect, the meloxicam plasma concentration is at least 1.0 μg/ml during a period from about 0 to 2 hours.

Various pharmaceutical compositions are contemplated for administration having a wide range of possible meloxicam solubilities. For example, in one aspect the composition may provide a meloxicam compound having a solubility in the composition of greater than about 1 mg/gm. In another aspect, the composition may provide a meloxicam compound having a solubility in the composition of greater than about 10 mg/gm. In yet another aspect, the composition may provide a meloxicam compound having a solubility in the composition of greater than about 50 mg/gm. Additionally, meloxicam compositions may be formulated with varying amounts of added water. For example, in one aspect the composition may include less than about 20% by weight of added water. In another aspect, the composition may be substantially non-aqueous.

Furthermore, the meloxicam compound may be included in the composition in a variety of forms. For example, in one aspect the meloxicam compound may be a meloxicam free acid. In another aspect, the meloxicam compound may be a meloxicam salt with a pharmaceutically acceptable counterion. In yet another example, the meloxicam compound may include a mixture of meloxicam free acid and a meloxicam salt with a pharmaceutically acceptable counterion having a weight ratio of meloxicam free acid to total meloxicam ranging from about 0.01 to about 0.99.

Various pharmaceutical compositions containing meloxicam are also contemplated. Such a composition may include a therapeutically effective amount of a meloxicam compound in a pharmaceutically acceptable carrier including at least one of an alkalizer or a solubilizer. In such a composition, the meloxicam compound may have a variety of solubilities. For example, in one aspect the meloxicam compound may have a solubility in the carrier that is greater than about 1.0 mg/gm. In another aspect, the meloxicam compound may have a solubility in the carrier that is greater than or equal to about 3.5 mg/gm. In yet another aspect, the meloxicam compound may have a solubility in the carrier that is greater than or equal to about 10 mg/gm. In a further aspect, the meloxicam compound may have a solubility in the carrier that is greater than or equal to about 50 mg/gm.

In addition to meloxicam as the active agent, the formulations of the present invention may include a solubilizer. A wide variety of solubilizers may be included in the formulations according to aspects of the present invention. In one aspect, the solubilizer may be a solvent, a polymer, or a mixture thereof. Additionally, in one aspect, the solubilizer may have a melting point that is less than about 80° C. In another aspect, the solublizer may be a nonaqueous liquid at a temperature of between about 32° C. and about 37° C.

Solvents are one potential class of solubilizer that may be included in the compositions of the present invention. Examples of such solvents may include, without limitation, polyoxyethylene sorbitan fatty acid esters, polyoxyethylene-polyoxypropylene block copolymers, polyglycerol fatty acid esters, polyoxyethylene glycerides, polyoxyehtylene sterols, deriviatives, and analogues thereof, polyoxyethylene vegetable oils, polyoxyethylene hydrogenated vegetable oils, reaction mixtures of polyols and at least one member of the group consisting of fatty acids, glycerides, vegetable oils, hydrogenated vegetable oils, and sterols, tocopheryl polyethylene glycol succinates, sugar esters, sugar ethers, sucroglycerides, alkylglucosides, alkylmaltosides, alkylthioglucosides, lauryl macrogolglycerides, polyoxyethylene alkyl ethers, polyoxyethylene alkylphenols, polyethylene glycol fatty acids esters, alkyl ammonium salts, salts of alkylsulfates, salts of fatty acids, sodium docusate, alcohols, polyols, ethers of polyethylene glycols, glycofurol, N-alkylpyrrolidone, 2-pyrrolidone, triacetin, dimethyl acetamide, dimethyl isosorbide and mixtures thereof.

Another potential class of solubilizers that may be useful when formulated with the compositions of the present inventions includes polymers. Examples of such polymers include, without limitation, high molecular weight polyethylene glycol, cellulosics (e.g., ethyl cellulose, methyl cellulose, hydroxypropyl cellulose, hydroxypropyl methyl cellulose, hydroxypropyl methyl cellulose phthalate, hydroxypropyl methyl cellulose succinate, hydroxypropyl cellulose, cellulose acetate, cellulose nitrate, cellulose acetate phthalate), polyethylene oxide, polyvinyl pyrrolodine, acrylic polymers (e.g., polyacrylic acid (CARBOMER®, neutral polymers of methacrylates (e.g., EUDRAGITNE), methacrylate copolymers with trimethylaminoethylmetacrylate as functional group (EUDRAGIT RS, RS 100, RL, RL100), anionic polymers of methacrylic acids and methacrylates (e.g., EUDRAGIT L 100, L 100-55, S100), high molecular weight polysachharide gums and resins (e.g., acacia, xanthan gum, tragacanth gum etc.), and combinations thereof.

Alkalizers can also be included in the pharmaceutical compositions of the present invention with or without a solublizer. In those aspects including both an alkalizer and a solubilizer in the composition, various ratios of alkalizer to solubilizer are contemplated. For example, in one aspect the weight ratio of alkalizer to solubilizer in the composition may range from about 0.005 to about 1.0. In another aspect, the weight ratio of alkalizer to solubilizer in the composition may range from about 0.02 to about 0.007. Examples of useful alkalizers may include, without limitation, amino acid, an amino acid ester, ammonium hydroxide, calcium hydroxide, potassium hydroxide, sodium hydroxide, sodium hydrogen carbonate, aluminum hydroxide, calcium carbonate, potassium carbonate, magnesium carbonate, magnesium hydroxide, methyl glucamine, diethanolamine, tromethamine, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, salts of a pharmaceutically acceptable cation and acetic acid, and combinations thereof. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium.

In one aspect, at least a portion of the meloxicam compound used in the present pharmaceutical compositions may be a solid meloxicam compound. For example, the composition may include solid meloxicam compound particles. In another aspect, the composition may comprise meloxicam compound in both a solubilized fraction and a solid fraction. A broad range of ratios of solubilized meloxicam compound to solid meloxicam compound particles may be used. For example, in one aspect the weight ratio of solubilized meloxicam compound to solid meloxicam compound particles can range from about 0.01 to about 0.99. In another aspect, the weight ratio of solubilized meloxicam compound to solid meloxicam compound particles ranges from about 0.2 to about 0.7. In one specific aspect, a solubilized fraction may contain meloxicam compound having a solubility in the composition of greater than about 1 mg/gm, and a solid fraction having solid meloxicam compound particles with an effective average diameter greater than 2.0 μm.

Additionally, the solid meloxicam particles may be present in a variety of forms. For example, in one aspect the solid meloxicam compound particles may have an effective average diameter greater than about 2.0 μm. In another aspect, the solid meloxicam compound particles may be formulated as a solid carrier. Though such a solid carrier may be in a variety of forms, non-limiting examples may include beads, beadlets, granules, spherules, pellets, microcapsules, microspheres, nanospheres, nanocapsules, tablets, or combinations thereof. Other forms known to those of ordinary skill in the art may also be used.

The compositions of the present invention may also be articulated in terms of meloxicam dissolution performance under certain specified conditions. For example, in one aspect of the present invention a pharmaceutical composition is provided including a therapeutically effective amount of meloxicam compound in combination with a pharmaceutically acceptable carrier that provides an amount of dissolved meloxicam greater than or equal to about 1.2 mg when dissolved in a USP type 2 apparatus at 100 rpm in a medium of 250 ml of 0.1 N hydrochloric acid at 37° C. In some aspects, the amount of dissolved meloxicam may be greater than or equal to about 2.5 mg.

In another aspect of the present invention, a pharmaceutical composition is provided that includes a therapeutically effective amount of meloxicam compound in combination with a pharmaceutically acceptable carrier which provides an amount of dissolved meloxicam greater than or equal to about 1.2 mg in less than 30 minutes after initiation of dissolution testing in a USP type 2 apparatus at 100 rpm in a dissolution medium of 250 ml of 0.1 N hydrochloric acid at 37° C. In another aspect, the amount of dissolved meloxicam may be greater than or equal to about 2.5 mg.

In yet another aspect, a pharmaceutical composition is provided which has a therapeutically effective amount of meloxicam compound combined with a pharmaceutically acceptable carrier that provides, upon in vitro dissolution in a USP type 2 apparatus at 100 rpm in a dissolution medium of 250 ml of 0.1 N hydrochloric acid at 37° C., an amount of dissolved meloxicam at one hour after the start of dissolution that is at least two times the amount of meloxicam compound dissolved at one hour from a comparative composition in which the meloxicam compound solubility in the carrier is less than 1 mg/gm.

The meloxicam compositions disclosed herein may be useful for treating or preventing pain and/or inflammation in a subject in need thereof. Such treatment may include perorally administering a composition as recited herein to the subject. Advantageously, in some aspects, the time to effective pain relief provided by the composition may be less than about 3 hours. In another aspect, the time to effective pain relief may be less than about 1 hour. In yet another aspect, the time to effective pain relief may be less than about 30 minutes.

Effective daily doses of the compositions of the present invention are also contemplated. For example, in one aspect, a total daily dose of a meloxicam compound may be less than or equal to about 15 mg. In another aspect, a total daily dose of a meloxicam compound may be less than 15 mg. In yet another aspect, a total daily dose of a meloxicam compound may be less than or equal to about 7.5 mg. In a further aspect, a total daily dose of a meloxicam compound may be less than about 7.5 mg.

In addition to the foregoing ingredients, in some aspects of the present invention the present compositions may also include a second, or additional active agent. Such second active agents may include nearly any useful active agent known to one of ordinary skill in the art. Examples include, without limitation, opioids, non-opioid analgesics such as ibuprofen, acetaminophen, aspirin, etc., cold or cough remedies, such as antihistamines, decongestants, expectorants, anti-tussives, 5-HT1 agonists, calcium channel blockers, beta-adrenergic receptor blocking agents, xanthine derivatives, prostaglandin analogs such as misoprostol, antacids, proton-pump inhibitors, and combinations thereof. The second or additional active agent may be formulated in the composition to be delivered according to nearly any desired release profile, such as immediate release or extended release. In those aspects where the second active agent is intended for extended release, the extended release may be sufficient to allow a therapeutically effective dose to be administered at 24 hour intervals.

### BRIEF DESCRIPTION OF FIGURES

FIG. 1 shows projected mean meloxicam plasma concentrations as a function of time after multiple dose administration of 15 mg meloxicam every 24 hours from the embodiment provided in Example 7-1 as compared to a commercial reference product MOBIC® (meloxicam) tablets

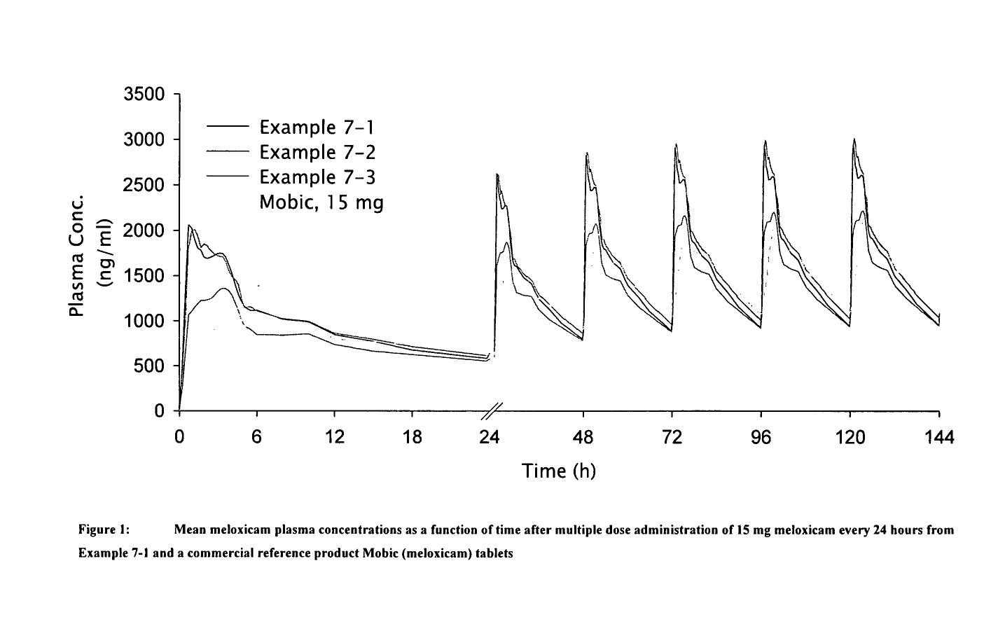

FIG. 2 shows pharmacokinetic/pharmacodynamic modeling of pain intensity as a function of time after single dose administration of 15 mg meloxicam in 12 subjects.

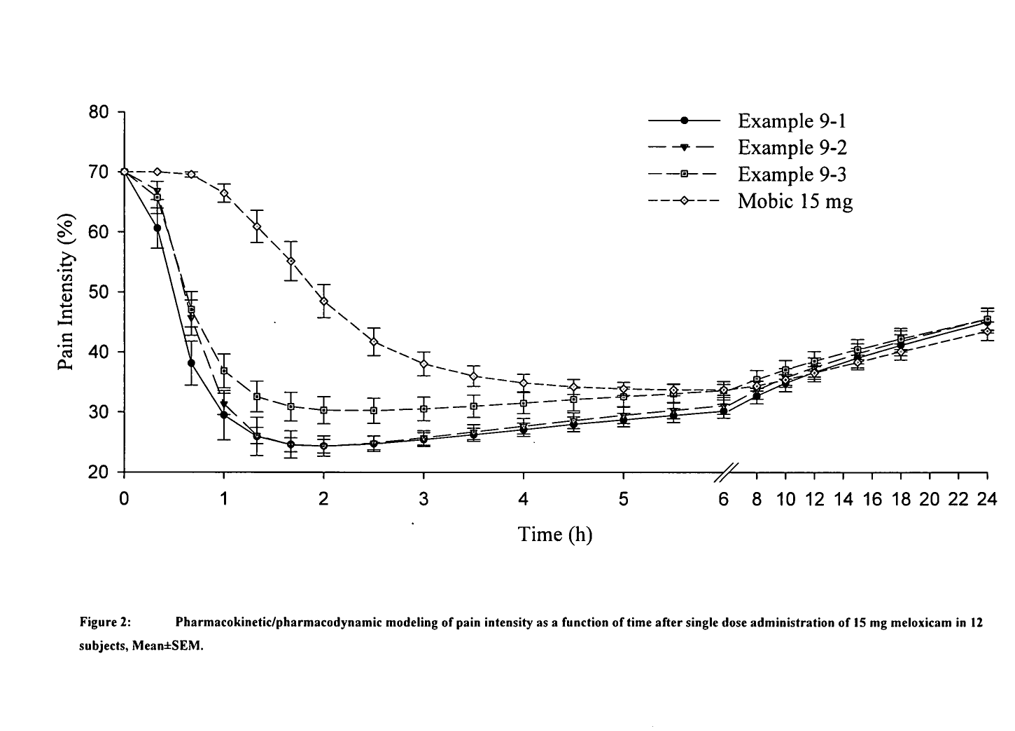

### DETAILED DESCRIPTION

Definitions

In describing and claiming the present invention, the following terminology will be used in accordance with the definitions set forth below.

The singular forms “a,” “an,” and, “the” include plural referents unless the context clearly dictates otherwise. Thus, for example, reference to “a drug” includes reference to one or more of such drugs, and reference to “an excipient” includes reference to one or more of such excipients.

As used herein, “active agent,” “bioactive agent,” “pharmaceutically active agent,” and “pharmaceutical,” may be used interchangeably to refer to an agent or substance that has measurable specified or selected physiologic activity when administered to a subject in a significant or effective amount. It is to be understood that the term “drug” is expressly encompassed by the present definition as many drugs and prodrugs are known to have specific physiologic activities. These terms of art are well-known in the pharmaceutical, and medicinal arts. Further, when these terms are used, or when a particular active agent is specifically identified by name or category, it is understood that such recitation is intended to include the active agent per se, as well as pharmaceutically acceptable, pharmacologically active derivatives thereof, or compounds significantly related thereto, including without limitation, salts, pharmaceutically acceptable salts, N-oxides, prodrugs, active metabolites, isomers, fragments, analogs, solvates hydrates, radioisotopes, etc.

As used herein, the terms “formulation” and “composition” are used interchangeably and refer to a mixture of two or more compounds, elements, or molecules. In some aspects the terms “formulation” and “composition” may be used to refer to a mixture of one or more active agents with a carrier or other excipients.

As used herein “meloxicam”, refers to a compound with the IUPAC name of (4-hydroxy-2-methyl-N-(5-methyl-2-thiazolyl-)-2H-1,2-benzothiazine-3-carboxamide 1,1-dioxide). Meloxicam is described in U.S. Pat. No. 4,233,299, which is hereby incorporated as a reference. Meloxicam is an oxicam derivative with the following chemical structure:

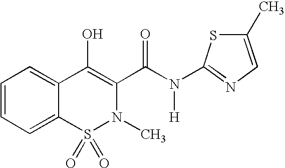

Meloxicam is practically insoluble in water and has low solubility in acidic or neutral mediums. Meloxicam is listed as monograph no. 5848 in The Merck Index 13^{th} ed., (2001), which is incorporated herein by reference.

As used herein, “meloxicam compound” and like terms refers to not only meloxicam, itself, but also includes meloxicam salts, meloxicam bases, meloxicam acids, prodrugs, metabolites, and combinations thereof.

As used herein, “subject” refers to a mammal that may benefit from the administration of a drug composition or method of this invention. Examples of subjects include humans, and may also include other animals such as horses, pigs, cattle, dogs, cats, rabbits, and aquatic mammals. In one specific aspect, a subject is a human.

As used herein, the terms “administration,” and “administering” refer to the manner in which an active agent is presented to a subject. Administration can be accomplished by various art-known routes such as oral, parenteral, transdermal, inhalation, implantation, etc.

The term “oral administration” represents any method of administration in which an active agent can be administered by swallowing, chewing, or sucking an oral dosage form. Such solid or liquid oral dosage forms are traditionally intended to substantially release and or deliver the active agent in the gastrointestinal tract beyond the mouth and/or buccal cavity. Examples of solid dosage forms include conventional tablets, capsules, caplets, etc., which do not substantially release the drug in the mouth or in the oral cavity.

As used herein, “oral dosage form” refers to a formulation that is prepared for administration to a subject through the oral route of administration. Examples of known oral dosage forms, include without limitation, tablets, capsules, caplets, powders, pellets, granules, solutions, suspensions, solutions and solution pre-concentrates, emulsions and emulsion pre-concentrates, etc. In some aspects, powders, pellets, and granules may be coated with a suitable polymer or a conventional coating material to achieve, for example, greater stability in the gastrointestinal tract, or to achieve the desired rate of release. Moreover, capsules containing a powder, pellets or granules may be further coated. Tablets and caplets may be scored to facilitate division of dosing. Alternatively, the dosage forms of the present invention may be unit dosage forms wherein the dosage form is intended to deliver one therapeutic dose per administration.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, “pharmaceutically acceptable carrier” and “carrier” may be used interchangeably, and refer to any inert and pharmaceutically acceptable material that has substantially no biological activity, and makes up a substantial part of the formulation.

The term “admixed” means that the drug and/or other ingredients can be dissolved, dispersed, or suspended in the carrier. In some cases, the drug may be uniformly admixed in the carrier.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

The compositions and methods of the present invention have as one of their objectives providing analgesia to a patient, and, in particular, to provide analgesia in a manner that achieves effective pain relief in a short period of time. Since pain reduction and pain intensity are not necessarily reflected in observable physical phenomena, it is common to use a patient reported outcome such as pain intensity or pain relief. These data can typically be reported either as a categorical or a continuous variable.

As an example of a categorical measure, pain intensity may be reported based on four categories: 0=No pain; 1=Mild or Slight Pain; 2=Moderate Pain, 3=Severe Pain. In this context, effective pain relief would indicate a reduction in the patient reported pain intensity of at least one category (for example, a change from severe pain at baseline to moderate pain). Another common categorical variable is the pain intensity difference (PID). Using the same pain intensity scale, the difference from the pain intensity at baseline and the pain intensity during treatment is calculated (ranging from −2 for a change from mild pain at baseline to severe pain on treatment to +3 for a change from severe pain at baseline to no pain on treatment). In this context, effective pain relief is to be defined as a PID value of +1 or greater.

Another common categorical variable is pain relief (PR), which is typically a patient reported variable with four categories: 0=none, 1=a little or slight relief, 2=a lot or moderate relief, and 4=complete relief. In this context, effective pain relief would represent a PR score of 1 or greater.

Often a composite variable (PRID) will be calculated from the sum of the PR and PBD scores. Using the scales described above, this variable would range from −3 (for no pain relief and a change in pain intensity from no pain at baseline to severe pain on treatment) to 7 (for complete pain relief and a difference in pain intensity from severe pain at baseline to no pain on treatment). Using this scale, effective pain relief would be indicated by a PRID score of +2 or greater.

Analgesic efficacy can also be assessed as a continuous variable using a patient reported outcome such as pain intensity or pain relief rated on a visual analog scale (VAS). Typically, for pain intensity the visual analog scale could consist of a 100 mm horizontal line measuring no pain at the left margin (0 mm) and severe pain or the worst possible pain at the right margin (100 mm). For pain relief, the visual analog scale could measure no pain relief at the left margin (0 mm) and complete pain relief at the right margin (100 mm). Using this measure, effective pain relief would be described by a reduction in the pain intensity score of at least 10 mm or a pain relief score of at least 10 mm.

Time to effective pain relief can be determined by repeated evaluations of pain intensity or pain relief using any standard measure. For example, the time to effective pain relief could be the amount of time between administration of the treatment and the observed time at which the PR score for a patient decreases by at least one category, the PID score is one or greater, the PRID score is 2 or greater, the VAS score for pain intensity decreases by 10 mm or more, or the VAS score for pain relief is at least 10 mm.

Other measures of time to effective pain relief which are in common use but which are not necessarily equivalent to the above definitions could also be used. For example, one common technique is to ask the patient to indicate (typically with a stopwatch) the time at which meaningful or effective pain relief occurs.

In addition to the goal of providing expedited analgesia to a subject, the present invention also aims to provide effective anti-inflammatory and anti-pyretic action in a reduced time frame as compared to known meloxicam formulations as well. Much research and energy has been expended in the study and evaluation of the body's inflammatory response and to mechanisms for the reduction and amelioration thereof.

A number of inflammation factors have been identified, such as aracidonic acid metabolites (prostaglandins and leukotrienes), bradykinin, complement proteins, histamine and serotonin, interleukins, including IL-1 and IL-8, platelet-activating factor (PAF) transforming growth factor (TGF) and tumor necrosis factors (TFN). One major class of anti-inflammatory is NSAIDS which, as mentioned above, inhibit prostaglandin synthesis by blocking the activity of the precursor enzyme, cylcooxygenase (COX). Those of ordinary skill in the art will understand various mechanisms for measuring reduction and prevention of inflammation, including reduced serum levels of several of the above-recited agents, improved movement and flexibility, reduced tissue tenderness, reduced redness and coloration, etc.

As is well know, pyretic response is the defensive mechanism of the body which elevates body temperature in response to the detection of an infection. The measurement of body temperature is easily obtained by use of various thermometer devices. Accordingly, one of ordinary skill in the art can readily detect and monitor the anti-pyretic effect of an active agent, including the time to onset, potency, etc.

As used herein, “maximum plasma concentration” and Cmax may be used interchangeably and refer to the maximum concentration of an active agent in the plasma of a subject which is achieved by administration of a formulation containing such active agent. Cmax is a generally known term in the pharmaceutical industry as a statistical indicator of peak drug plasma concentration. It is know that Cmax can be used to identify peak drug plasma concentration over any desired therapy duration. That is to say, that Cmax can be used to identify peak drug plasma concentration over the entire duration of a pharmacotherapy regimen, or for selected segments thereof. Accordingly, such terms may be used herein in connection with time identifiers such as hours, days, minutes, etc., to indicate the maximum, or peak, plasma concentrations occurring during such segments.

As used herein, the term “about” is used to provide flexibility to a numerical range endpoint by providing that a given value may be “a little above” or “a little below” the endpoint.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

Concentrations, amounts, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually.

This same principle applies to ranges reciting only one numerical value as a minimum or a maximum. Furthermore, such an interpretation should apply regardless of the breadth of the range or the characteristics being described.

The Invention

In order to obtain more effective pain relief and control of inflammation, the inventors have discovered a pharmaceutical composition that may provide a more rapid, uniform, and/or predictable release of meloxicam as compared with current formulations. Such improvements in release may reduce the time to reach effective plasma concentrations of meloxicam, and thus may decrease the time to onset of analgesic and/or anti-inflammatory efficacy for orally administered meloxicam. Achieving decreased times to effective plasma concentrations and rapid onset of activity without significantly increasing the maximum plasma concentration of the meloxicam may also improve both the efficacy and safety of meloxicam formulations. In addition to the treatment of pain and inflammation, the formulations of the present invention may be used to treat or prevent any of the indications recited herein. Accordingly, the treatments of such indications are also to be considered within the present scope.

Aspects of the present invention provide rapidly absorbing meloxicam compounds from pharmaceutical compositions to subjects that more rapidly provide associated therapeutic effects. In one aspect of the present invention, a method of providing meloxicam therapy to a subject is provided that may include perorally administering to the subject a therapeutically effective amount of a meloxicam compound from a composition that provides a meloxicam plasma concentration during the period from about 0 to 1 hours after administration which is at least about 40% of the maximum plasma concentration attained by the composition. In another aspect, the meloxicam plasma concentration during a period from about 0 to 1 hours after administration is at least about 80% of the maximum plasma concentration attained by the composition. In some aspects, the maximum plasma concentration may be the maximum plasma concentration observed between about 2 hours and about 10 hours after administration of the formulation. In yet another aspect, the meloxicam plasma concentration may be at least 1.0 μg/ml during a period from about 0 to 2 hours.

Various aspects of the present invention may also be characterized in terms of meloxicam dissolution under certain specified conditions. It should be noted that the term “meloxicam dissolution” is intended to describe the amount of a meloxicam compound dissolved under the specified parameters outlined herein rather than the rate of dissolution. As such, in one aspect of the present invention a pharmaceutical composition is provided including a therapeutically effective amount of a meloxicam compound in a pharmaceutically acceptable carrier that provides an amount of dissolved meloxicam greater than or equal to about 1.2 mg when dissolved in a USP type 2 apparatus at 100 rpm in a medium of 250 ml of 0.1 N hydrochloric acid at 37° C. In other words, at least about 2.5 mg of a meloxicam compound is dissolved in 250 ml of 0.1 N hydrochloric acid at 37° C. under the specified conditions. In another aspect, the amount of dissolved meloxicam may be greater than or equal to about 2.5 mg.

In another aspect of the present invention, a pharmaceutical composition is provided including a therapeutically effective amount of a meloxicam compound in a pharmaceutically acceptable carrier that provides an amount of dissolved meloxicam greater than or equal to about 1.2 mg in less than 30 minutes after initiation of dissolution testing in a USP type 2 apparatus at 100 rpm in a dissolution medium of 250 ml of 0.1 N hydrochloric acid at 37° C. In other words, at least about 1.2 mg of a meloxicam compound is dissolved in 250 ml of 0.1 N hydrochloric acid at 37° C. under the specified conditions in less than 30 minutes following initiation of the dissolution procedure. However, in some aspects, the solubility may be greater, and the amount of dissolved meloxicam may be greater than or equal to about 2.5 mg.

In yet another aspect, a pharmaceutical composition is provided including a therapeutically effective amount of a meloxicam compound in a pharmaceutically acceptable carrier that provides, upon in vitro dissolution in a USP type 2 apparatus at 100 rpm in a dissolution medium of 250 ml of 0.1 N hydrochloric acid at 37° C., an amount of dissolved meloxicam at one hour after the start of dissolution that is at least two times the amount of meloxicam compound dissolved at one hour from a comparative composition in which the meloxicam compound solubility in the carrier is less than 1 mg/gm. In other words, following dissolution under the specified conditions, at least two times the amount of a meloxicam compound is dissolved in 250 ml of 0.1 N hydrochloric acid at 37° C. as compared to a composition having meloxicam solubility of less than 1 mg/gm.

Various solubilities may be achieved by the pharmaceutical compositions according to aspects of the present invention. Meloxicam compositions can be formulated having varying solublities to provide dosage forms that may be administered in a wide variety of circumstances. For example, very highly concentrated dosage forms containing higher doses of a meloxicam compound can be formulated for once-a-day formulations, or for those subjects that tolerate meloxicam well and are experiencing high levels of pain or inflammation. Dosage forms that are less concentrated and thus contain lower doses of a meloxicam compound can be formulated for multiple daily dosing, or for those individuals that experience meloxicam side effects at higher doses or are experiencing lower levels of pain or inflammation. By way of example without limitation, formulations according to the present invention may in some aspects be formulated for administration to a subject one time per day. In another aspect, the formulations may be formulated for administration more than one time per day. In yet another aspect, the formulation may be suitable for administration two, three, or even 4 or more times per day.

Furthermore, in one aspect, the composition may provide a meloxicam compound having a solubility in the composition or the carrier of greater than about 1 mg/gm. In another aspect, the composition may provide a meloxicam compound having solubility in the composition or the carrier that is greater than or equal to about 3.5 mg/gm. In yet another aspect, the composition may provide a meloxicam compound having a solubility in the composition or the carrier of greater than about 10 mg/gm. In a further aspect, the composition may provide a meloxicam compound having a solubility in the composition or the carrier of greater than about 50 mg/gm.

Aspects of the present invention also may provide rapidly absorbing meloxicam compounds from pharmaceutical compositions to subjects more rapidly provide associated therapeutic effects. In one aspect of the present invention, a method of providing meloxicam therapy to a subject is provided that may include perorally administering to the subject a therapeutically effective amount of a meloxicam compound from a composition that provides a meloxicam plasma concentration at about 40 minutes which is at least about 40% of the maximum plasma concentration attained by the formulation. In another aspect, the meloxicam concentration at about 20 minutes is at least about 40% of the maximum plasma concentration attained by the formulation. In another aspect, the meloxicam plasma concentration at about 40 minutes is at least about 80% of the maximum plasma concentration attained by the formulation. In some aspects, such maximum meloxicam plasma concentrations may be the maximum concentration observed between about 2 hours and about 10 hours after administration of the formulation. In yet another aspect, the meloxicam plasma concentration may be at least 1.0 μg/ml after about 40 minutes following single-dose administration. It should be noted that it is not intended that the measurement of Tₘₐₓ include blood plasma concentrations associated with enterohepatic recycling.

Various aspects of the present invention also may provide meloxicam compositions that have low amounts of added water as compared to previous formulations. In one aspect, for example, the composition may include less than about 20% by weight of added water. In another aspect, the composition may be substantially nonaqueous. It is important to note that the above recited solubilities may be achieved in some aspects with compositions that are substantially nonaqueous or that contain very little added water.

The pharmaceutical compositions according to the various aspects of the present invention can be formulated to provide dosage forms that contain a highly solubilized meloxicam compound for rapid absorption and decreased Tₘₐₓ in a subject. For example, in one aspect a composition may include a therapeutically effective amount of a meloxicam compound in a pharmaceutically acceptable carrier including an alkalizer, where the meloxicam compound has a solubility in the carrier that is greater than about 1.0 mg/gm. In another aspect, a composition may include a therapeutically effective amount of a meloxicam compound in a pharmaceutically acceptable carrier including a solubilizer, where the meloxicam compound has a solubility in the carrier that is greater than about 1.0 mg/gm. In yet another aspect, a composition may include a therapeutically effective amount of a meloxicam compound in a pharmaceutically acceptable carrier including both an alkalizer and a solubilizer, where the meloxicam compound has a solubility in the carrier that is greater than about 1.0 mg/gm.

Meloxicam can be included in the formulation in any form known to one of ordinary skill in the art. A particular form of meloxicam can thus be selected for inclusion in a composition that provides high solubility and rapid absorption. In one aspect, for example, meloxicam can be included in the formulation as a meloxicam free acid. In another aspect, meloxicam can be included in the formulation as a meloxicam salt with a pharmaceutically acceptable counterion. In yet another aspect, meloxicam can be included in the formulation as a mixture of meloxicam free acid and a meloxicam salt with a pharmaceutically acceptable counterion having a weight ratio of meloxicam free acid to total meloxicam ranging from about 0.01 to about 0.99. Counterions for inclusion with the meloxicam salt may include any pharmaceutically acceptable counterion known to one of ordinary skill in the art. Exemplary couterions may include, without limitation, amino acid, an amino acid ester, ammonium hydroxide, calcium hydroxide, potassium hydroxide, sodium hydroxide, sodium hydrogen carbonate, aluminum hydroxide, calcium carbonate, potassium carbonate, magnesium carbonate, magnesium hydroxide, methyl glucamine, diethanolamine, tromethamine, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, salts of a pharmaceutically acceptable cation and acetic acid, and combinations thereof. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium.

In various aspects of the present invention, the composition may also be formulated to provide a meloxicam compound in immediate, pulsatile, delayed, or sustained release forms. Compositions may also be formulated to provide a meloxicam compound in any combination of immediate, pulsatile, delayed, or sustained release forms. For example, in one aspect a single composition may be formulated to provide a meloxicam compound as both immediate and sustained release. Such a formulation would allow immediate and sustained therapeutic effects with a single dose and thus reduce problems associated with dosage forms requiring more frequent administration.

The amount of a meloxicam compound to be orally administered may be measured according to several different parameters. In one aspect, the amount of the meloxicam compound administered may be an amount sufficient to achieve a therapeutic effect. The amount required to obtain a therapeutic effect may vary depending on a number of factors, including the activity or potency of the specific meloxicam formulation, as well as physiological variations among subjects as to drug tolerance and general metabolic issues. In one aspect, behavioral variation can provide some measure of therapeutic effectiveness. As such, it is well within the knowledge of those skilled in the art and in view of the present disclosure to determine dosages of the meloxicam compound that are therapeutically effective for a given subject. Accordingly, any therapeutically effective amount of a meloxicam compound known to one of ordinary skill in the art can be formulated into a single dosage. Such dosage forms may be intended for once-a-day or more frequent administration. In one aspect, for example, a single dose may be formulated to contain less than or equal to about 15 mg of meloxicam. In another aspect, a single dose may be formulated to contain less than 15 mg of meloxicam. In yet another example, a single dose may be formulated to contain less than or equal to about 7.5 mg of meloxicam. In a further aspect, a single dose may be formulated to contain less than 7.5 mg of meloxicam. It should be noted that the formulated concentration of meloxicam may vary somewhat depending on the form of meloxicam, i.e. meloxicam free acid, meloxicam salt, meloxicam free acid and salt, etc. It should also be noted that dosages may be highly variable depending on the potency of the meloxicam formulation, and as such, the previously disclosed dosages are not to be limiting in any way.

Varying amounts of meloxicam compounds can be formulated into dosage forms having a particular volume. For example, in one aspect, up to 30 mg of a meloxicam compound having a solubility in the carrier of greater than about 50 mg/gm may be formulated into a dosage form having a volume of less than 0.6 cm³. In another aspect, up to 15 mg of a meloxicam compound having a solubility in the carrier of greater than about 50 mg/gm may be formulated into a dosage form having a volume of less than 0.3 cm³. In yet another aspect, up to 7.5 mg of a meloxicam compound having a solubility in the carrier of greater than about 50 mg/gm may be formulated into a dosage form having a volume of less than 0.15 cm³.

The pharmaceutical compositions of the present invention can be incorporated in oral dosage forms such as a particles, beads, capsules, tablets, etc. In one aspect, the oral dosage form may be a capsule or tablet. In another embodiment the oral dosage form may include a multi-component dosage form such as beads in a capsule, a capsule or capsules in a capsule, a tablet or tablets in a capsule, or a multilayer tablet.

In one aspect, the composition of the present invention may be formulated as a pre-concentrate, such as an “emulsion pre-concentrate” or a “micro-emulsion pre-concentrate”. Such formulations have been traditionally shown useful for the efficacious delivery of highly water insoluble drugs. That is to say, that upon administration to a subject such compositions form a solution, suspension, emulsion, micro-emulsion, or undergo an other type of conversion in vivo that improves the bioavailability and absorption of the active agent. One example of such formulations may be found in U.S. Pat. No. 5,342,625, which is incorporated herein by reference. Other examples of pre-concentrate formulations may be found in U.S. Pat. Nos. 6,267,984; 6,294,192; and 6,451,339, each of which is incorporated herein by reference. Other solutions, solutions pre-concentrates, and suspensions will be recognized by those of ordinary skill in the art as improving in vivo absorption for a variety of active agents.

As has been described, specific aspects of the present invention may include a solubilizer to, inter alia, increase the solubility of the meloxicam compound in the carrier. In one aspect, it may be beneficial to include a solubilizer in the pharmaceutical composition that has a melting point less than about 80° C. In another aspect, it may be beneficial to include a solubilizer in the pharmaceutical composition that is a nonaqueous liquid at a temperature of between about 32° C. and about 37° C. Additionally, though any solubilizer that can effectively solubilize the meloxicam compound to the degree specified herein can be utilized in compositions according to aspects of the present invention, specific class examples may include solvents, polymers, and mixtures thereof.

As has been described, a solvent may be used to solubilize the meloxicam compound in various aspects of the present invention. Any solvent capable of solublizing the meloxicam compound to the extent described in aspects of the present invention may be utilized in the present pharmaceutical formations. Examples include, without limitation, polyoxyethylene sorbitan fatty acid esters, polyoxyethylene-polyoxypropylene block copolymers, polyglycerol fatty acid esters, polyoxyethylene glycerides, polyoxyehtylene sterols, deriviatives, and analogues thereof, polyoxyethylene vegetable oils, polyoxyethylene hydrogenated vegetable oils, reaction mixtures of polyols and at least one member of the group consisting of fatty acids, glycerides, vegetable oils, hydrogenated vegetable oils, and sterols, tocopheryl polyethylene glycol succinates, sugar esters, sugar ethers, sucroglycerides, alkylglucosides, alkylmaltosides, alkylthioglucosides, lauryl macrogolglycerides, polyoxyethylene alkyl ethers, polyoxyethylene alkylphenols, polyethylene glycol fatty acids esters, alkyl ammonium salts, salts of alkylsulfates, salts of fatty acids, sodium docusate, alcohols, polyols, ethers of polyethylene glycols, glycofurol, N-alkylpyrrolidone, 2-pyrrolidone, triacetin, dimethyl acetamide, dimethyl isosorbide and mixtures thereof. Examples of specific low-melting and/or liquid solvents may include, without limitation, alcohols, polyols, ethers of polyethylene glycols, glycofurol, N-alkylpyrrolidone, 2-pyrrolidone, triacetin, dimethyl acetamide, dimethyl isosorbide and mixtures thereof.

Various polymers may also be utilized to solubilize the meloxicam compound in various aspects of the present invention. Any polymer capable of solublizing the meloxicam compound to the extent described in aspects of the present invention may be utilized in the present pharmaceutical formations. Examples include, without limitation, high molecular weight polyethylene glycol, cellulosics (e.g., ethyl cellulose, methyl cellulose, hydroxypropyl cellulose, hydroxypropyl methyl cellulose, hydroxypropyl methyl cellulose phthalate, hydroxypropyl methyl cellulose succinate, hydroxypropyl cellulose, cellulose acetate, cellulose nitrate, cellulose acetate phthalate), polyethylene oxide, polyvinyl pyrrolodine, acrylic polymers (e.g., polyacrylic acid (CARBOMER®, neutral polymers of methacrylates, (e.g., EUDRAGIT NE), methacrylate copolymers with trimethylaminoethylmetacrylate as functional group (EUDRAGIT RS, RS 100, RL, RL100), anionic polymers of methacrylic acids and methacrylates (e.g., EUDRAGIT L 100, L 100-55, S100), high molecular weight polysachharide gums and resins (e.g., acacia, xanthan gum, tragacanth gum etc.), and combinations thereof.

As has been described, various alkalizers can be utilized to solubilize a meloxicam compound in various aspects of the present invention. An alkalizer can be used alone, or in combination with a solubilizer such as a solvent or a polymer. The relative amounts of the alkalizer and the solubilizer in the pharmaceutical composition may vary depending on the particular nature of the composition. In one aspect, however, the weight ratio of alkalizer to solubilizer in the composition may range from about 0.005 to about 1.0. In another aspect, the weight ratio of alkalizer to solubilizer in the composition may range from about 0.02 to about 0.007.

Any alkalizer capable of solublizing a meloxicam compound to the extent described in aspects of the present invention may be utilized in the present pharmaceutical formations. Non-limiting examples of alkalizers may include amino acid, an amino acid ester, ammonium hydroxide, calcium hydroxide, potassium hydroxide, sodium hydroxide, sodium hydrogen carbonate, aluminum hydroxide, calcium carbonate, potassium carbonate, magnesium carbonate, magnesium hydroxide, methyl glucamine, diethanolamine, tromethamine, magnesium aluminum silicate, synthetic aluminum silicate, synthetic hydrotalcite, magnesium aluminum hydroxide, diisopropylethylamine, ethanolamine, ethylenediamine, triethanolamine, triethylamine, triisopropanolamine, salts of a pharmaceutically acceptable cation and acetic acid, and combinations thereof. Also suitable are bases which are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Preferred cations include sodium, potassium, lithium, magnesium, calcium and ammonium.

The inventors have also discovered that beneficial pharmaceutical compositions can be formulated to include a solubilized fraction of a meloxicam compound and a solid fraction of the meloxicam compound. In one aspect, the solid fraction may include solid meloxicam compound particles. Such particles may be milled, micronized, etc. Such formulations may increase the absorption rate and provide additional flexibility with regard to release profiles. The solid fraction may be in contact with the solubilized fraction in the composition, or it may be formulated separately from the solubilized fraction. In one aspect, the weight ratio of solubilized meloxicam compound to solid meloxicam compound particles may range from about 0.01 to about 0.99. In another aspect, weight ratio of solubilized meloxicam compound to solid meloxicam compound particles may range from about 0.2 to about 0.7.

In those aspects incorporating a solid fraction of meloxicam compound, various size ranges for the solid meloxicam compound particles are contemplated. For example, in one aspect, solid meloxicam compound particles may have an effective average diameter greater than about 2.0 μm. Additionally, in one aspect, the solid fraction may be formulated as beads, beadlets, granules, spherules, pellets, microcapsules, microspheres, nanospheres, nanocapsules, tablets, or combinations thereof. Such solid fraction particles may be include in the composition with or without an exterior coating. Such coatings may be utilized to further alter the release profile of the solid fraction, to protect the solid fraction, etc.

Various active agents in addition to meloxicam may also be formulated in the pharmaceutical compositions of the present invention. Such active agents may include any active agent that can be beneficially coadministered with the meloxicam compound. Various configurations of meloxicam and a second active agent are contemplated. For example, in one aspect the meloxicam compound and second active agent can be formulated separately and included in the same pharmaceutical composition. In another aspect, the meloxicam compound and the second active agent can be co-formulated for inclusion in the pharmaceutical composition. Whether formulated separately from the meloxicam compound or co-formulated with the meloxicam compound, the second active agent can be formulated for controlled release, including immediate-release, rapid-onset, sustained-release, extended release, pulsatile release, and/or dual-release form. In one aspect, such a second active agent may be acidic, with pH-dependent solubility. In another aspect, a second active agent may include an opioid and/or another analgesics, including narcotic analgesics, Mu receptor antagonists, Kappa receptor antagonists, non-narcotic (i.e., non-addictive) analgesics, monoamine uptake inhibitors, adenosine regulating agents, cannabinoid derivatives, cannabinoid receptor modulators, vanilloid receptor modulators, Substance P antagonists, neurokinin-1 receptor antagonists and sodium channel blockers, and the like, as well as combinations thereof.

Non-limiting examples of second active agents may include aceclofenac, acemetacin, e-acetamidocaproic acid, acetaminophen, acetaminosalol, acetanilide, acetylsalicylic acid (aspirin), S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, bendazac, benorylate, benoxaprofen, benzpiperylon, benzydamine, benzylmorphine, bermoprofen, bezitramide, -bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butophanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac sodium, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, diprocetyl, dipyrone, ditazol, droxicam, emorfazone, enfenamic acid, epirizole, eptazocine, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, isofezolac, isoladol, isomethadone, isonixin, isoxepac, isoxicam, ketobemidone, ketoprofen, ketorolac, p-lactophenetide, lefetamine, levorphanol, lofentanil, lonazolac, lomoxicam, loxoprofen, lysine acetylsalicylate, magnesium acetylsalicylate, meclofenamic acid, mefenamic acid, meperidine, meptazinol, mesalamine, metazocine, methadone hydrochloride, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, piprofen, pirazolac, piritramide, piroxicam, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalte, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, zomepirac, and combinations thereof. Specific opioids that may be particularly useful when administered in combination with meloxicam may include, without limitation, codeine, meperidine, fentanyl, methadone, d-propoxyphene, pentazocine, buprenorphine, naloxone, naltrexone, dextromethorphan, morphine, tramadol, nalmefene, or combinations thereof.

In yet another aspect of the present invention, meloxicam compound compositions can be formulated in combination with COX-2 inhibitors. Examples of COX-2 inhibitors may include, without limitation, celecoxib, rofecoxib (VIOXX®), valdecoxib, parecoxib, MK-966 (Merk), etoricoxib (MK-663; Merk), SC-236 (4-[5-(4-chlorophenyl)-3-(trifluoromethyl)-1H-pyrazol-1-yl)]benzenesulfonamide; G.D. Searle & Co.); NS-398 (N-(2-cyclohexyloxy-4-nitrophenyl)methane sulfonamide; Taisho Pharmaceutical Co., Ltd., Japan); SC-58125 (methyl sulfone spiro(2.4)hept-5-ene I; Pharmacia/Searle & Co.); SC-57666 (Pharmacia/Searle & Co.); SC-58635 (celexcoxib; Pharmacia/Searle & Co.); SC-558 (Pharmacia/Searle & Co.); SC-560 (Pharmacia/Searle & Co.); etodolac (LODINE®, Wyeth-Ayerst Laboratories, Inc.); DFU (5,5-dimethyl-3-(3-fluorophenyl)-4-(4-methylsulfonyl)phenyl 2(5H)-furanone); MK-476, L-745337, L-761066, L-761000, L-748780, and L-748731 (Merck); DUP-697 (5-Bromo-2-(4-fluorophenyl)-3-(4-(met-hylsulfonyl)phenyl; DuPont Merck Pharmaceutical Co.); PGV 20229 (1-(7-tert.-butyl-2,3-dihydro-3,3-dimethylbenzo(b)furan-5-yl)-4-cyclopropylbutan-1-one) (Procter & Gamble Pharmaceuticals); T-614 (3-formylamino-7-methylsulfonylamino-6-phenoxy-4H-1-benzopyran-4-one; Toyama Corp., Japan); BF 389 (Biofor, USA); PD 136005, PD 142893, and PD 145065 (all Parke-Davis/Warner-Lambert Co.); flurbiprofen (ANSAID®; Pharmacia & Upjohn); nimesulide (NIM-03, Mesulid®; Hisamitsu, Japan); nabumetone (RELAFEN®; SmithKline Beecham, plc); flosulide (CGP 28238; Novartis/Ciba Geigy); piroxicam (FELDENE®; Pfizer); dicofenac (VOLTAREN® and CATAFLAM®, Novartis); COX-189 (Novartis); D 1367 (Celltech Chiroscience, plc); R 805 (4 nitro 2 phenoxymethane sulfonanilide); R 807 (3 benzoyldifluoromethane sulfonanilide, diflumidone); JTE-522 (Japan Tobacco, Japan); FK-3311 (4′-Acetyl-2′-(2,4-difluorophenoxy)methanesulfonanilide; Fujisawa, Japan); FK 867 (Fujisawa, Japan); FR 115068 (Fujisawa, Japan); GR 253035 (Glaxo Wellcome); RWJ 63556 (Johnson & Johnson); RWJ 20485 (Johnson & Johnson); ZK 38997 (Schering); S 2474 ((E)-(5)-(3,5-di-tert-butyl-4-hydro-xybenzylidene)-2-ethyl-1,2-isothiazolidine-1,1-dioxide indomethacin; Shionogi & Co., Ltd., Japan); CL 1004 (Parke-Davis); RS 57067 (Hoffmann La Roche); RS 104894 (Hoffmann La Roche); SC 41930 (Monsanto); SB 205312 (SmithKline Beecham); SKB 209670 (SmithKline Beecham, plc); and Ono 1078 (Ono Pharmaceutical Co., Japan).

In a further aspect of the present invention, meloxicam compositions may be administered in combination with a cold and cough remedies such as antihistamines, decongestants, antitussives, expectorants, etc. Examples of antihistamines may include, without limitation, bromodiphenhydramine, brompheniramine, carbinoxamine, chlorpheniramine, dexchlorpheniramine, diphenhydramine, doxylamine, phenindamine, pheniramine, phenyltoloxamine, pyrilamine, promethazine, and triprolidine. Non-limiting examples of decongestants may include ephedrine, phenylephrine, pseudoephedrine, and combinations thereof. Examples of antitussives may include, without limitation, narcotics such as codeine, dihydrocodeine, hydrocodone, hydromorphone, etc., or non-narcotics such as carbetapentane, caramiphen, dextromethorphan, etc. One example of an expectorant antihistamines may include, without limitation, guaifenesin. In another specific example, a meloxicam compound composition may also be administered in combination with a sedative.

One specific example indication where a combination therapy of a meloxicam compound and a second active agent may be beneficial includes painful headache or migraine conditions. Such painful conditions may be effectively treated by a combination of meloxicam with a vasomodulator such as a xanthine derivative. An example of a xanthine derivative may include an alkylxanthine compound. The term “alkylxanthine” herein is intended to include, without limitation, xanthine derivatives having one or more C₁₋₄ alkyl substituents and pharmaceutically acceptable salts of such xanthine derivatives. Specific non-limiting examples may include dimethylxanthines and trimethylxanthines, including caffeine, theobromine, and theophylline. In another aspect, the meloxicam composition may be administered in combination with a 5-HT1 agonist such as a triptan for the treatment of migraine or other indication. Exemplary 5-HT1 agonists may include sumatriptan, frovatripta, zolmitriptan, eletriptan, riztriptan, almotriptan, naratriptan, etc., or combinations thereof.

The pharmaceutical compositions according to aspects of the present invention may also include various compounds in the formulation that may not be considered active agents, but are provided to exert an effect on the subject. One example of such a compound may include an antacid, such as those disclosed in U.S. Patent Application No. 2005/38018, which is herein incorporated by reference. The amount of antacid in the oral pharmaceutical dosage form may vary depending on the intended usage and the type of antacid. In one aspect, however, from about 10 mg to about 7000 mg of antacid may be included in the composition. In another aspect, from about 16 mg to about 4000 mg of antacid may be included in the composition.

Various aspects of the present invention may also include methods or treatment. For example, in one aspect a method of treating pain in a subject is provided. Such a method may include perorally administering to the subject a therapeutically effective amount of meloxicam from a composition that provides a time to effective pain relief of less than about 3 hours. In another aspect, the time to effective pain relief is less than about 1 hour. In yet another aspect, the time to effective pain relief is less than about 30 minutes.

### EXAMPLES

The following examples of formulations of meloxicam formulations are provided to promote a more clear understanding of certain embodiments of the present invention, and are in no way meant as a limitation thereon.

### Example 1

TABLE 1
Composition of solubilized meloxicam systems

| | Composition(% w/w) | | | | | |
|---|---|---|---|---|---|---|
| | Example | Example | Example | Example | Example | Example |
| Ingredient | 1-1 | 1-2 | 1-3 | 1-4 | 1-5 | 1-6 |
| Meloxicam | 2.3 | 2.2 | 2.0 | 2.0 | 2.2 | 2.2 |
| Polyoxyl 40 | 96.6 | 95.2 | 95.2 | — | 85.2 | |
| hydrogenated castor oil | | | | | | |
| Tromethamine | 1.1 | 1.1 | — | 1.1 | 1.1 | 1.1 |
| Meglumine | — | — | 1.1 | — | — | — |
| Vitamin E | — | 1.5 | 1.5 | 1.5 | 1.5 | — |
| Polyoxyl 35 castor oil | — | — | — | 95.4 | | |
| Polyethylene glycol | — | — | — | — | 10.0 | |
| 4000 | | | | | | |
| Ethanol | — | — | — | — | — | 10 |
| Propylene glycol | — | — | — | — | — | 76.7 |
| Polysorbate 80 | — | — | — | — | — | 10 |
| | Composition(% w/w) | | | | | |
| | Example | Example | Example | Example | Example | Example |
| Ingredient | 1-7 | 1-8 | 1-9 | 1-10 | 1-11 | 1-12 |
| Meloxicam | 2.3 | 2.2 | 2.2 | 2.0 | 2.0 | 2.2 |
| Polyoxyl 40 | 95.4 | — | 95.0 | 22.8 | — | 20 |
| hydrogenated castor oil | | | | | | |
| Tromethamine | 0.73 | 1.1 | — | 1.1 | 1.1 | 1.1 |
| Vitamin E | 1.5 | 1.5 | 1.5 | 1.5 | 1.5 | 1.1 |
| Polyoxyl 35 castor oil | — | 71 | | 63.1 | — | 22 |
| Polyethylene glycol | — | 24.2 | — | — | — | 15 |
| 4000 | | | | | | |
| Ethanol | — | — | — | — | 5 | 12.5 |
| Propylene glycol | — | — | — | 5 | 65 | 10 |
| Polysorbate 80 | — | — | — | 4.5 | 25.4 | 16.1 |
| Sodium hydroxide | — | — | 0.23 | — | — | — |

Meloxicam is dissolved in a mixture of polyoxyl 40 hydrogenated castor oil or polyoxyl 35 castor oil and alkalyzer. Other additives such vitamin E and polyethylene glycol 4000 were added and mixed to get solution. The solution is filled into 2 piece hard gelatin capsules having 15 mg of meloxicam per capsule as shown in Table 1.

Dissolution is carried out in 250 ml of simulated gastric fluid (SGF; pH 1.2) at 37° C. using USP type 2 apparatus operated at 100 rpm. The samples are withdrawn and filtered through 0.2 micron Nylon filter and are analyzed for meloxicam content by liquid chromatography. The in vitro dissolution results shown in Table 2 demonstrate that the composition of Table 1 has significantly enhanced dissolution as compared to control. The control composition is a commercially available tablet, MOBIC® (meloxicam) tablets, 15 mg; Boehringer Ingelheim.

TABLE 2
In vitro dissolution of meloxicam compositions in SGF (pH 1.2) at 37° C.

| | Dissolved meloxicam in Mg (Mean ± SD) | | | | |
|---|---|---|---|---|---|
| | | Example | Example | | Example |
| Time (min) | Control | 1-1 | 1-4 | Example 1-5 | 1-2 |
| 2 | 0.13 ± 0.007 | 10.51 ± 1.0 | 12.24 ± 1.11 | 1.56 ± 0.4 | 8.8 ± 0.8 |
| 15 | 0.18 ± 0.007 | 4.81 ± 1.1 | 13.38 ± 0.38 | 3.78 ± 0.4 | 7.7 ± 0.8 |
| 30 | 0.20 ± 0.002 | 2.28 ± 0.6 | 8.79 ± 0.24 | 2.20 ± 0.2 | 6.7 ± 0.6 |
| 60 | 0.24 ± 0.003 | 1.63 ± 0.4 | 5.69 ± 0.59 | 1.56 ± 0.1 | 4.5 ± 0.03 |

### Example 2

TABLE 3
Meloxicam compositions: combination of solubilized systems and solid granules

| | Composition(mg/unit) | | | | |
|---|---|---|---|---|---|
| | Example | Example | Example | Example | Example |
| Ingredient | 2-1 | 2-2 | 2-3 | 2-4 | 2-5 |
| Component A | | | | | |
| (Solubilized system) | | | | | |
| Meloxicam | 7.5 | 7.5 | 7.5 | 7.5 | 7.5 |
| Polyoxyl 40 hydrogenated | 307.0 | | 311.0 | — | — |
| castor oil | | | | | |
| Polyoxyl 35 castor oil | — | 307.0 | — | 311.0 | — |
| Polyethylene glycol 400 | | | | | 311.0 |
| Tromethamine | 5.0 | 5.0 | 5.0 | 5.0 | 5.0 |
| Vitamine E | 4.0 | 4.0 | — | — | — |
| Component B (Granules) | | | | | |
| Meloxicam (micronised) | 7.50 | 7.50 | | 7.50 | 7.50 |
| Melxoicam (nanosized) | | | 7.50 | | |
| Lactose Monohydrate | 63.0 | 63.0 | 63.0 | 63.0 | 63.0 |
| Microcrystalline Cellulose | 21.0 | 21.0 | 21.0 | 21.0 | 21.0 |
| Colloidal Silicon Dioxide | 0.50 | 0.50 | 0.50 | 0.50 | 0.50 |
| Sodium Citrate Dihydrate | 7.50 | 7.50 | 7.50 | 7.50 | 7.50 |
| Magnesium Stearate | 0.50 | 0.50 | 0.50 | 0.50 | 0.50 |

Meloxicam is micronized to a particle size of less than 10 μm (90%) or nanosized to get particle size of less than 2 μm (90%) and is mixed with other excipients. The granules are filled into 2 piece hard gelatin capsules. Meloxicam is dissolved in a mixture of surfactant (polyoxy 35 castor oil, polyethylene glycolm, polyoxy 40 hydrogenated castor oil), alkalyzer and vitamin E. The solution is filled into capsules and then the capsules containing the meloxicam granules are inserted into the solution. The total meloxicam dose unit is about 15 mg. Dissolution is performed as described in Example 1 and is outlined in Table 4.

TABLE 4
In vitro dissolution of meloxicam compositions
in SGF (pH 1.2) at 37° C.

Dissolved meloxicam in Mg
(Average ± SD)

| Time | Control | Example 2-1 |
|---|---|---|
| 5 | 0.13 ± 0.007 | 3.49 ± 0.25 |
| 15 | 0.18 ± 0.007 | 3.89 ± 0.25 |
| 30 | 0.20 ± 0.002 | 2.64 ± 0.14 |
| 60 | 0.24 ± 0.003 | 1.76 ± 0.05 |

The in vitro dissolution test shows that the composition shown in Table 3 has significantly enhanced dissolution as compared to the control, Mobic® (meloxicam) tablets, 15 mg; Boehringer Ingelheim.

### Example 3

TABLE 5
Meloxicam tablets

| | Composition(% w/w) | | | | | |
|---|---|---|---|---|---|---|
| | Example | Example | Example | Example | Example | Example |
| Ingredient | 3-1 | 3-2 | 3-3 | 3-4 | 3-5 | 3-6 |
| Meloxicam | 1.3 | — | 1.3 | 0.65 | 0.8 | 1.3 |
| (micronised) | | | | | | |
| Meloxicam (nanosized) | — | 1.3 | — | 0.65 | 0.5 | |
| Cremophor RH40 | 12.5 | 12.5 | | 12.5 | 6 | 11.3 |
| Tocopherol | — | — | 12.5 | — | 6.5 | 1.2 |
| polyethylene glycol | | | | | | |
| 1000 succinate | | | | | | |
| Lactose | 17.1 | 17.1 | 17.1 | 17.1 | 13.59 | 17.8 |
| Microcrystalline | 51.75 | 51.75 | 51.75 | 51.75 | 53.4 | 51.75 |
| cellulose | | | | | | |
| Ethocel 10 cps | 1 | 1 | 1 | 1 | 1 | 1 |
| Sodium starch glycolate | 8 | 8 | 8 | 8 | 9.2 | — |
| Croscarmellose Sodium | — | — | — | — | — | 7.3 |
| BHT | 0.1 | 0.1 | 0.1 | 0.1 | 0.1 | 0.1 |
| Sodium Citrate | 1.5 | 1.5 | 1.5 | 1.5 | 0.86 | 1.5 |
| Dihydrate | | | | | | |
| PVP K30 | 6 | 6 | 6 | 6 | 7.3 | 6 |
| Magnesium Stearate | 0.25 | 0.25 | 0.25 | 0.25 | 0.25 | 0.25 |
| Talc | 0.5 | 0.5 | 0.5 | 0.5 | 0.5 | 0.5 |

Meloxicam is micronized to achieve a particle size of less than about 10 μm (90%) or nanosized to get particle size of less than about 2 μm (90%) and is mixed with other excipients. The resulting powder is granulated using a combination of ethanol and water mixture, and is dried at about 40° C. The granules are compressed into a tablet by using compression machine. The dose of meloxicam per unit is about 15 mg. Dissolution testing is performed as described in Example 1 and is outlined in Table 6.

TABLE 6
In vitro dissolution of meloxicam compositions (example 3-1)
in SGF (pH 1.2) at 37° C.

Dissolved meloxicam
in Mg (Average ± SD)

| Time | Control | Example 3-1 |
|---|---|---|
| 5 | 0.13 ± 0.007 | 0.88 ± 0.12 |
| 15 | 0.18 ± 0.007 | 1.27 ± 0.01 |
| 30 | 0.20 ± 0.002 | 1.21 ± 0.04 |
| 60 | 0.24 ± 0.003 | 1.18 ± 0.04 |

The in vitro dissolution test shows that the composition shown in Table 5 has significantly enhanced dissolution as compared to the control tablet, Mobic® (meloxicam) tablets, 15 mg; Boehringer Ingelheim.

### Example 4

TABLE 7
Composition of meloxicam solubilized systems

| | Composition(% w/w) | | | | | |
|---|---|---|---|---|---|---|
| | Example | Example | Example | Example | Example | Example |
| Ingredient | 4-1 | 4-2 | 4-3 | 4-4 | 4-5 | 4-6 |
| Meloxicam | 3.0 | 4.0 | 2.0 | 2.2 | 2.2 | 3.0 |
| Polyoxyl 40 hydrogenated | 94.1 | 93.0 | — | — | 66.64 | 94.1 |
| castor oil | | | | | | |
| Polyethylene glycol | — | — | 94.4 | — | — | — |
| Polyoxyl 35 castor oil | — | — | — | 23.8 | — | — |
| Polyethylene glycol - 8 | — | — | — | — | 28.56 | — |
| Caprylic/capric glycerides | | | | | | |
| Caprylic/capric glycerides | — | — | — | 47.6 | — | — |
| Tocopherol polyethylene | — | — | — | 23.8 | — | — |
| glycol 1000 succinate | | | | | | |
| Tromethamine | 1.5 | 1.5 | 1.1 | 1.1 | 1.1 | — |
| Vitamin E | 1.5 | 1.5 | 1.5 | 1.5 | 1.5 | 1.5 |
| Sodium hydroxide | — | — | — | — | — | 0.23 |

Surfactants/solvents (Polyoxyl 40 hydrogenated castor oil, polyethylene glycol, polyoxyl 35 castor oil) and other optional components are mixed and the drug is dissolved. The solution is filled into 2 piece hard gelatin capsules having 15 mg of meloxicam per capsule. The release of meloxican from the composition is given in Table 8.

TABLE 8
In vitro dissolution of meloxicam compositions
in SGF (pH 1.2) at 37° C.

| | Dissolved meloxicam in Mg (Mean ± SD) | | | |
|---|---|---|---|---|
| | | Example | Example | Example |
| Time | Control | 4-2 | 4-1 | 4-3 |
| 5 | 0.13 ± 0.007 | 3.00 ± 0.39 | 7.54 ± 0.54 | 5.46 ± 0.27 |
| 15 | 0.18 ± 0.007 | 1.75 ± 0.07 | 2.43 ± 0.33 | 3.86 ± 0.48 |
| 30 | 0.20 ± 0.002 | 0.13 ± 0.007 | 1.61 ± 0.07 | 3.33 ± 0.60 |
| 60 | 0.24 ± 0.003 | 0.18 ± 0.007 | 1.51 ± 0.01 | 2.85 ± 0.56 |

### The in vitro dissolution test shows that the composition according to Example 4 has significantly enhanced dissolution until 60 minutes as compared to control.

### Example 5

TABLE 9
Solubilized meloxicam compositions with added water

| | Composition | | | | |
|---|---|---|---|---|---|
| | (% w/w) | | | | |
| | Example | Example | Example | Example | Example |
| Ingredient | 6-1 | 6-2 | 6-3 | 6-4 | 6-5 |
| Meloxicam | 2.2 | 2.0 | 2.0 | 2.0 | 2.2 |
| Polyoxyl 40 hydrogenated | 89.2 | 89.4 | 89.4 | 89.4 | 90.0 |
| castor oil | | | | | |
| Tromethamine | 1.1 | 1.1 | 1.1 | 1.1 | 1.1 |
| Sodium hydroxide | — | — | — | — | 0.23 |
| Vitamin E | 1.5 | 1.5 | 1.5 | 1.5 | 1.5 |
| Polyoxyl 35 castor oil | | 89.2 | — | — | |
| Polyethylene glycol 4000 | — | | — | — | — |
| Ethanol | — | — | | — | — |
| Polysorbate 80 | — | — | | | — |
| Water | 5.0 | 5.0 | 5.0 | 5.0 | 5.0 |

Solubilizers (polyoxyl 40 hydrogenated castor oil or polyoxyl 35 castor oil or polyethylene glycol or ethanol or polysorbate 80), tromethamine and vitamin E are mixed with water as given in table 9 of example 5 and the drug is dissolved. The solution is filled into 2 piece hard gelatin capsules having 15 mg of meloxicam per capsules. The release of meloxicam from the composition is given in Table 12.

TABLE 10
In vitro dissolution of meloxicam compositions
in SGF (pH 1.2) at 37° C.

Dissolved meloxicam
in Mg (Mean ± SD)

| Time | Control | Example 6-1 |
|---|---|---|
| 5 | 0.13 ± 0.007 | 9.76 ± 0.28 |
| 15 | 0.18 ± 0.007 | 11.67 ± 2.69 |
| 30 | 0.20 ± 0.002 | 6.32 ± 2.84 |
| 60 | 0.24 ± 0.003 | 2.27 ± 0.5 |

### The in vitro dissolution test shows that the composition according to Example 6 has significantly enhanced dissolution until 60 minutes as compared to control.

### Example 6

TABLE 11
Compositions of solubilized meloxicam systems
with varying meloxicam concentration

| | Composition (% w/w) | | | |
|---|---|---|---|---|
| | Example | Example | Example | Example |
| Ingredient | 8-1 | 8-2 | 8-3 | 8-4 |
| Meloxicam | 3.0 | 4.05 | 3.0 | 4.05 |
| Polyoxyl 35 Castor oil | 93.45 | 91.16 | 95.1 | — |
| Polyethylene glycol | — | — | — | 95.1 |
| 400 | | | | |
| Tromethamine | 2.05 | 2.76 | — | — |
| Vitamin E | 1.5 | 2.025 | 1.5 | 1.5 |
| Sodium hydroxide | — | — | 0.34 | 0.34 |

Polyoxyl 35 Castor oil or polyethylene glycol 400 and other optional components are mixed and the drug is dissolved. The solution is filled into 2 piece hard gelatin capsules having 15 mg of meloxicam per capsules. The release of meloxicam from the composition is given in Table 12.

TABLE 12
In vitro dissolution of meloxicam compositions
in SGF (pH 1.2) at 37° C.

| | Dissolved meloxicam in Mg (Mean ± SD) | | |
|---|---|---|---|
| Time | Control | Example 8-1 | Example 8-2 |
| 5 | 1.7 ± 0.1 | 9.1 ± 1.2 | 4.15 ± 0.47 |
| 15 | 2.4 ± 0.1 | 3.6 ± 0.7 | 2.08 ± 0.11 |
| 30 | 2.7 ± 0.3 | 2.3 ± 0.4 | 1.57 ± 0.04 |
| 60 | 3.2 ± 0.04 | 1.7 ± 0.2 | 1.34 ± 0.05 |

### The in vitro dissolution test shows that the composition according to Example 8 has significantly enhanced dissolution over 60 minutes as compared to control.

### Example 7

The dosage forms of Examples 1 and 2 were dosed in a randomized, single-dose crossover study in healthy volunteers with a conventional immediate release dosage form as a comparator (Reference; Mobic® (meloxicam) tablets, 15 mg; Boehringer Ingelheim) as follows:

| | |
|---|---|
| Example 7-1: | Example 1-2, 15 mg meloxicam/capsule |
| Example 7-2: | Example 1-5, 15 mg meloxicam/capsule |
| Example 7-3: | Example 2-1, 15 mg meloxicam/capsule |
| Reference: | Mobic ® (meloxicam) tablet, 15 mg |

Venous blood samples (6 ml) were serially collected by direct venipuncture into plastic K₂EDTA Vacutainer® collection tubes. Plasma was separated using standard procedures and assayed for meloxicam using a validated LC/MS/MS method.

Twelve subjects completed treatments with Example 13-1 and the reference product and data from these subjects were analyzed using standard pharmacokinetic analysis techniques with WinNonLin Professional Version 5.0.0 (Pharsight Corporation, 800 West El Camino Real, Suite 200, Mountain View, Calif. 94040).

Meloxicam plasma concentrations for all three of the dosage forms prepared according to the current invention achieved very high levels (>1,000 ng/ml) in less than one hour for essentially all subjects. Plasma concentrations then plateaued during the period between about 2-10 hours, and finally entered a first-order elimination phase from about 24 hours on with a mean half life of 17-20 h.

As a measure of the rate with which meloxicam plasma concentrations reached maximum therapeutic levels, the ratio of the plasma concentrations at each time point, Cₚ(t) to the maximum observed plasma concentration, Cₘₐₓ, was calculated and the results are summarized in Table 13.

TABLE 13

| | Cₚ/Cₘₐₓ | | | |
|---|---|---|---|---|
| | Mean ± SEM | | | |
| Time (h) | Example 7-1 | Example 7-2 | Example 7-3 | Reference |
| 0.33 | 37 ± 10% | 18 ± 6% | 20 ± 7% | 5 ± 1% |
| 0.67 | 86 ± 7% | 73 ± 6% | 69 ± 9% | 13 ± 3% |
| 1.00 | 84 ± 6% | 90 ± 4% | 74 ± 7% | 21 ± 4% |
| 1.33 | 77 ± 6% | 90 ± 3% | 77 ± 6% | 27 ± 5% |
| 1.67 | 76 ± 6% | 85 ± 3% | 80 ± 5% | 37 ± 6% |
| 2.0 | 73 ± 4% | 87 ± 3% | 79 ± 4% | 48 ± 6% |
| 2.5 | 74 ± 4% | 84 ± 3% | 81 ± 2% | 62 ± 7% |
| 3.0 | 77 ± 4% | 80 ± 3% | 86 ± 5% | 71 ± 7% |
| 3.5 | 76 ± 4% | 80 ± 3% | 89 ± 3% | 84 ± 6% |
| 4.0 | 69 ± 3% | 73 ± 3% | 86 ± 4% | 85 ± 6% |

After administration of the dosage forms prepared according to the present invention, the plasma concentrations by 40 minutes in essentially all subjects were at or near the Cₘₐₓ. In contrast, meloxicam plasma concentrations from the conventional dosage form were only 14% of the Cₘₐₓ and rose very slowly thereafter, reflecting the slow absorption of meloxicam using conventional technology. After administration of the conventional tablet, subjects typically did not reach a stable plateau concentration until about 3 hours (range 2.5 hours to 10 hours). As with the investigational products, all subjects had entered a first-order elimination phase by 24 hours after administration of the reference product, with a mean first-order elimination half-life of 18 hours.

Plasma concentrations after multiple dose administration were simulated for the dosage form of Example 7-1 and the comparative reference product using simple non-compartmental linear superposition [WinNonLin Professional Version 5.0.0; Pharsight Corporation]. The projected plasma concentrations as a function of time are shown in Error! Reference source not found.. The results show that the reference product prepared using conventional technology reaches steady state after about 3-4 days with mean peak concentrations of about 2,000 ng/ml. In contrast, the example prepared according to the present invention at the same dose reaches a mean peak meloxicam plasma concentration of about 2,000 ng/ml within less than 1 hour of the first dose on the first day.

The multiple dose administration results also show that even at steady state the investigational dosage form prepared according to the present invention reaches peak or near peak concentrations by less than 1 hour after administration. In contrast, the conventional dosage form does not approach peak concentrations until about 4 hours after each dose administration at steady state.

Pharmacodynamic analgesic effects of meloxicam can be modeled assuming an indirect response model [Dayneka et al. J. Pharmacol. Kinet. Biopharm. 21:457-478 (1993)], in which the rate of change in the pain response variable is governed by the following differential equation

$$\frac{d R}{d t} = {k}_{i n} - {k}_{out} \cdot R$$

where R is the pain response (%), kᵢₙ is the zero-order rate constant for production of the response (%/h), and kₒᵤₜ is the first-order rate constant for loss of the response (h⁻¹). The effect of meloxicam on the pain response in an inflammatory pain model has been described by Giraudel et al. with sigmoid Iₘₐₓ inhibition of the production of the response as follows:

$$\frac{d R}{d t} = {k}_{i n} \cdot ( 1 - \frac{{I}_{max} \cdot {C}_{p}^{n}}{I {C}_{50}^{n} + {C}_{p}^{n}} ) - {k}_{out} \cdot R$$

where Iₘₐₓ is the maximum inhibition attributed to the drug, IC50 is the meloxicam concentration producing half maximum inhibition (883 ng/ml), n is a slope parameter for the shape of the sigmoid response (n=10), kᵢₙ and kₒᵤₜ are as described above (366%/h and 3.72 h−1, respectively, derived from a pain score using a radiant heat analgesia meter IITC/Life Science Model 390) [Giraudel et al. British Journal of Pharmacology 146:642-653 (2005)]. For the purposes of simulation, the pain score was converted to a %response on a visual analog scale with an assumption of maximum pain intensity scores of 70% based on clinical data obtained from use of meloxicam in treatment of acute sciatica [Auvinet et al. Clinical Therapeutics 17:1078-1090 (1990)]. With this conversion, the kᵢₙ is estimated at 260%/h and the other parameters are unchanged.

For performing the pharmacokinetic/pharmacodynamic modeling, the pharmacokinetic data were fit to a simple 2-compartment model with first-order absorption and a lag time. The pharmacokinetic data were well-described by this simplified model (correlation coefficient>0.9).

Results of the pharmacodynamic simulation for the single dose case are shown in Error! Reference source not found. All three of the dosage forms prepared according to the present invention rapidly achieve effective pain relief and maintain a level of pain relief comparable to or better than the comparative reference product for at least 24 hours. The predicted PID (pain intensity difference) scores and the proportion of subjects expected to achieve effective pain relief (PID of at least −10%) at each timepoint are summarized in Table 14 and Table 15, respectively.

TABLE 14

| | PID [PI − PIₜ₌₀] | | | |
|---|---|---|---|---|
| | Mean ± SEM | | | |
| Time | Example 7-1 | Example 7-2 | Example 7-3 | Reference |
| 0 | 0 ± 0% | 0 ± 0% | 0 ± 0% | 0 ± 0% |
| (pre-dose) | | | | |
| 20 | −9 ± 3% | −3 ± 2% | −4 ± 3% | 0 ± 0% |
| 40 | −32 ± 4% | −24 ± 3% | −23 ± 3% | 0 ± 0% |
| 60 | −41 ± 4% | −39 ± 2% | −33 ± 3% | −4 ± 1% |
| 80 | −44 ± 3% | −44 ± 1% | −37 ± 3% | −9 ± 3% |
| 100 | −45 ± 2% | −45 ± 1% | −39 ± 2% | −15 ± 3% |
| 120 | −46 ± 2% | −46 ± 1% | −40 ± 2% | −22 ± 3% |
| 150 | −45 ± 1% | −45 ± 1% | −40 ± 2% | −28 ± 2% |
| 180 | −45 ± 1% | −44 ± 1% | −39 ± 2% | −32 ± 2% |
| 210 | −44 ± 1% | −43 ± 1% | −39 ± 2% | −34 ± 2% |
| 240 | −43 ± 1% | −42 ± 1% | −38 ± 2% | −35 ± 1% |
| 270 | −42 ± 1% | −41 ± 1% | −38 ± 2% | −36 ± 1% |
| 300 | −41 ± 1% | −41 ± 1% | −37 ± 2% | −36 ± 1% |
| 330 | −41 ± 1% | −40 ± 1% | −37 ± 2% | −36 ± 1% |
| 360 | −40 ± 1% | −39 ± 1% | −36 ± 2% | −36 ± 1% |

TABLE 15

| | Proportion of Subjects with Effective Pain Relief (%) | | | |
|---|---|---|---|---|
| Time | Example 7-1 | Example 7-2 | Example 7-3 | Reference |
| 0 | 0% | 0% | 0% | 0% |
| (pre-dose) | | | | |
| 20 | 36% | 10% | 11% | 0% |
| 40 | 91% | 90% | 89% | 0% |
| 60 | 91% | 100% | 100% | 9% |
| 80 | 100% | 100% | 100% | 45% |
| 100 | 100% | 100% | 100% | 64% |
| 120 | 100% | 100% | 100% | 91% |
| 150 | 100% | 100% | 100% | 100% |
| 180 | 100% | 100% | 100% | 100% |

Approximately 90% of subjects are expected to achieve effective pain relief within 40 minutes after administration of the dosage forms prepared according to the current invention. For the comparative reference product in this PK/PD model, none of the subjects would be expected to achieve effective pain relief in 40 minutes after administration and only 45% of the subjects are expected to achieve effective pain relief by 80 minutes.

It is to be understood that the above-described compositions and modes of application are only illustrative of preferred embodiments of the present invention. Numerous modifications and alternative arrangements may be devised by those skilled in the art without departing from the spirit and scope of the present invention and the appended claims are intended to cover such modifications and arrangements. Thus, while the present invention has been described above with particularity and detail in connection with what is presently deemed to be the most practical and preferred embodiments of the invention, it will be apparent to those of ordinary skill in the art that numerous modifications, including, but not limited to, variations in size, materials, shape, form, function and manner of operation, assembly and use may be made without departing from the principles and concepts set forth herein.

What is claimed is:

1. A method of providing meloxicam therapy to a subject, comprising:
perorally administering to the subject a therapeutically effective amount of a meloxicam compound from a composition that provides a meloxicam plasma concentration during a period from about 0 to about 1 hours after administration which is at least about 40% of the maximum plasma concentration (Cmax) attained by the formulation.

2. The method of claim 1, wherein the meloxicam plasma concentration during a period from about 0 to 1 hours after administration is at least about 80% of the maximum plasma concentration (Cmax) attained by the formulation.

3. The method of claim 1, wherein the maximum plasma concentration is a maximum plasma concentration observed between about 2 hours and about 10 hours after administration.

4. The method of claim 1, wherein meloxicam plasma concentration is at least 1.0 μg/ml during a period from about 0 to about 2 hours.

5. The method of claim 4, wherein the period is from about 0 to about 1 hour.

6. The method of claim 1, wherein the amount of the meloxicam compound administered is a dose of meloxicam that is less than or equal to about 30 mg.

7. The method of claim 1, wherein the amount of the meloxicam compound administered is a dose of meloxicam that is less than or equal to about 15 mg.

8. The method of claim 1, wherein the amount of the meloxicam compound administered is a dose of meloxicam that is less than or equal to about 7.5 mg.

9. The method of claim 1, wherein the administration of meloxicam is a single dose administration.

10. The method of claim 9, wherein the single dose administration occurs once-daily.

11. The method of claim 1, wherein the administration of meloxicam is a multiple dose administration achieving steady state meloxicam plasma concentration.

12. The method of claim 1, wherein the composition provides a meloxicam compound having a solubility in the composition of greater than about 1 mg/gm.

13. The method of claim 1, wherein the composition provides a meloxicam compound having a solubility in the composition of greater than about 10 mg/gm.

14. The method of claim 1, wherein the composition provides a meloxicam compound having a solubility in the composition of greater than about 50 mg/gm.

15. The method of claim 1, wherein the composition includes less than about 20% by weight of added water.

16. The method of claim 1, wherein the composition is substantially nonaqueous.

17. The method of claim 1, wherein the composition comprises solid meloxicam compound particles.

18. The method of claim 10, wherein the composition further comprises a solid fraction having solid meloxicam compound particles.

19. The method of claim 1, wherein the composition comprises a liquid dosage form.

20. The method of claim 19, wherein the liquid dosage form is selected from the group consisting essentially of: solutions, solution pre-concentrates, suspensions, emulsions, emulsions pre-concentrates, and micro-emulsion pre-concentrates.

21. The method of claim 1, wherein the meloxicam compound is a meloxicam free acid.

22. The method of claim 1, wherein the meloxicam compound comprises a meloxicam salt with a pharmaceutically acceptable counterion.

23. The method of claim 1, wherein the meloxicam compound comprises a mixture of a meloxicam free acid and a meloxicam salt with a pharmaceutically acceptable counterion having a weight ratio of meloxicam free acid to total meloxicam ranging from about 0.01 to about 0.99.

24. A pharmaceutical composition, comprising:
a therapeutically effective amount of a meloxicam compound in a pharmaceutically acceptable carrier, said carrier including at least one of an alkalizer or a solubilizer,
said meloxicam compound having a solubility in the carrier that is greater than about 1.0 mg/gm.

25. The pharmaceutical composition of claim 24, wherein the meloxicam compound has a solubility in the carrier that is greater than or equal to about 3.5 mg/gm.

26. The pharmaceutical composition of claim 24, wherein the meloxicam compound has a solubility in the carrier that is greater than or equal to about 10 mg/gm.

27. The pharmaceutical composition of claim 24, wherein the meloxicam compound has a solubility in the carrier that is greater than or equal to about 50 mg/gm.

28. The pharmaceutical composition of claim 24, wherein the composition further comprises a solid fraction having solid meloxicam compound particles.

29. The pharmaceutical composition of claim 28, wherein a ratio of solubilized meloxicam compound to solid meloxicam compound particles ranges from about 0.01 to about 0.99.

30. The pharmaceutical composition of claim 28, wherein a ratio of solubilized meloxicam compound to solid meloxicam compound particles ranges from about 0.2 to about 0.8.

31. The pharmaceutical composition of claim 28, wherein the solid meloxicam compound particles have an effective average diameter greater than about 2.0 μm.

32. The pharmaceutical composition of claim 28, wherein the solid meloxicam compound particles are formulated as a solid carrier.

33. The pharmaceutical composition of claim 32, wherein the solid carrier includes a member selected from the group consisting of beads, beadlets, granules, spherules, pellets, microcapsules, microspheres, nanospheres, nanocapsules, tablets, or combinations thereof.

34. The pharmaceutical composition of claim 24, comprising up to 30 mg of meloxicam compound formulated into a dosage form, wherein the dosage form has a volume of less than 0.7 cm³.

35. The pharmaceutical composition of claim 24, comprising up to 15 mg of meloxicam compound formulated into a dosage form, wherein the dosage form has a volume of less than about 0.35 cm³.

36. The pharmaceutical composition of claim 24, comprising up to 7.5 mg of meloxicam compound formulated into a dosage form, wherein the dosage form has a volume of less than about 0.15 cm³.

37. The pharmaceutical composition of claim 24, wherein the composition includes less than about 20% by weight of added water.

38. The pharmaceutical composition of claim 24, wherein the composition is substantially nonaqueous.

39. The pharmaceutical composition of claim 24, wherein the composition comprises a liquid dosage form.

40. The pharmaceutical composition of claim 39, wherein the liquid dosage form is selected from the group consisting essentially of: solutions, solution pre-concentrates, suspensions, emulsions, emulsions pre-concentrates, and micro-emulsion pre-concentrates.

41. The pharmaceutical composition of claim 24, wherein the solubilizer has a melting point less than about 45° C.

42. The pharmaceutical composition of claim 41, wherein the solubilizer is a nonaqueous liquid at a temperature of between about 32° C. and about 37° C.

43. The pharmaceutical composition of claim 24, wherein the solubilizer is selected from the group consisting of solvents, polymers, and mixtures thereof.

44. The pharmaceutical composition of claim 43, wherein the solubilizer is a solvent.

45. The pharmaceutical composition of claim 44, wherein the solvent includes a member selected from the group consisting of polyoxyethylene sorbitan fatty acid esters, polyoxyethylene-polyoxypropylene block copolymers, polyglycerol fatty acid esters, polyoxyethylene glycerides, polyoxyehtylene sterols, deriviatives, and analogues thereof, polyoxyethylene vegetable oils, polyoxyethylene hydrogenated vegetable oils, reaction mixtures of polyols and at least one member of the group consisting of fatty acids, glycerides, vegetable oils, hydrogenated vegetable oils, and sterols, tocopheryl polyethylene glycol succinates, sugar esters, sugar ethers, sucroglycerides, alkylglucosides, alkylmaltosides, alkylthioglucosides, lauryl macrogolglycerides, polyoxyethylene alkyl ethers, polyoxyethylene alkylphenols, polyethylene glycol fatty acids esters, alkyl ammonium salts, salts of alkylsulfates, salts of fatty acids, sodium docusate, alcohols, polyols, ethers of polyethylene glycols, glycofurol, N-alkylpyrrolidone, triacetin, dimethyl acetamide, dimethyl isosorbide and mixtures thereof.

46. The pharmaceutical composition of claim 43, wherein the solubilizer is a polymer.

47. The pharmaceutical composition of claim 46, wherein the polymer includes a member selected from the group consisting of high molecular weight polyethylene glycol; cellulosics such as ethyl cellulose, methyl cellulose, hydroxypropyl cellulose, hydroxypropyl methyl cellulose, hydroxypropyl methyl cellulose phthalate, hydroxypropyl methyl cellulose succinate, hydroxypropyl cellulose, cellulose acetate, cellulose nitrate, and cellulose acetate phthalate; polyethylene oxide; polyvinyl pyrrolodine; acrylic polymers such as polyacrylic acid; neutral polymers of methacrylates; methacrylate copolymers with trimethylaminoethylmetacrylate as a functional group; anionic polymers of methacrylic acids and methacrylates; high molecular weight polysachharide gums and resins such as acacia, xanthan gum, and tragacanth gum, and mixtures thereof

48. The pharmaceutical composition of claim 24, wherein the composition includes an alkalizer selected from the group consisting of amino acid, an amino acid ester, ammonium hydroxide, calcium hydroxide, potassium hydroxide, sodium hydroxide, sodium hydrogen carbonate, aluminum hydroxide, calcium carbonate, potassium carbonate, magnesium carbonate, magnesium hydroxide, methyl glucamine, diethanolamine, tromethamine, magnesium aluminum silicate, synthetic aluminum silicate, synthetic hydrotalcite, magnesium aluminum hydroxide, diisopropylethylamine, ethanolamine, ethylenediamine, triethanolamine, triethylamine, and triisopropanolamine, salts of a pharmaceutically acceptable cation and acetic acid, salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, and uric acid, salts of polyprotic acids, such as sodium phosphate, disodium hydrogen phosphate, sodium dihydrogen phosphate, and combinations thereof.

49. The pharmaceutical composition of claim 24, wherein the composition further includes an alkalizer and a solubilizer.

50. The pharmaceutical composition of claim 49, wherein a weight ratio of alkalizer to solubilizer in the composition ranges from about 0.005 to about 1.0.

51. The pharmaceutical composition of claim 49, wherein a ratio of alkalizer to solubilizer in the composition ranges from about 0.02 to about 0.7.

52. A pharmaceutical composition, comprising:
a therapeutically effective amount of a meloxicam compound in a pharmaceutically acceptable carrier that provides an amount of dissolved meloxicam greater than or equal to about 1.2 mg when dissolved in a USP type 2 apparatus at 100 rpm in a medium of 250 ml of 0.1 N hydrochloric acid at 37° C.

53. A pharmaceutical composition, comprising:
a therapeutically effective amount of a meloxicam compound in a pharmaceutically acceptable carrier that provides an amount of dissolved meloxicam greater than or equal to about 1.2 mg in less than 120 minutes after initiation of dissolution testing in a USP type 2 apparatus at 100 rpm in a dissolution medium of 250 ml of 0.1 N hydrochloric acid at 37° C.

54. The composition of claim 53, wherein the amount of dissolved meloxicam is greater than or equal to about 1.2 mg in less than about 60 minutes after initiation of dissolution.

55. The composition of claim 53, wherein the amount of dissolved meloxicam is greater than or equal to about 1.2 mg in less than about 30 minutes after initiation of dissolution.

56. The composition of claim 53, wherein the amount of dissolved meloxicam is greater than or equal to about 1.2 mg in less than about 10 minutes after initiation of dissolution.

57. A pharmaceutical composition, comprising:
a therapeutically effective amount of a meloxicam compound in a pharmaceutically acceptable carrier that provides, upon in vitro dissolution in a USP type 2 apparatus at 100 rpm in a dissolution medium of 250 ml of 0.1 N hydrochloric acid at 37° C., an amount of dissolved meloxicam at 1 hour after the start of dissolution that is at least two times the amount of meloxicam dissolved at 1 hour from a comparative composition in which the meloxicam solubility in the carrier is less than 1 mg/gm.

58. The composition of either of claims 53 or 57, wherein the meloxicam compound is meloxicam free acid.

59. The composition of either of claims 53 or 57, wherein the meloxicam compound comprises a meloxicam salt with a pharmaceutically acceptable counterion.

60. The composition of either of claims 53 or 57, wherein the meloxicam compound comprises a mixture of meloxicam free acid and a meloxicam salt with a pharmaceutically acceptable counterion, in which the ratio of meloxicam free acid to total meloxicam ranges from about 0.01 to about 0.99.

61. A method of treating pain in a subject, comprising perorally administering to the subject a therapeutically effective amount of a meloxicam compound from a composition that provides a time to effective pain relief of less than about 2 hours.

62. The method of claim 61, wherein the time to effective pain relief is less than about 1 hour.

63. The method of claim 61, wherein the time to effective pain relief is less than about 30 minutes.

64. The method of claim 61, wherein a total daily dose of the meloxicam compound is less than or equal to about 15 mg.

65. The method of claim 64, wherein the total daily dose of the meloxicam compound is less than 15 mg.

66. The method of claim 61, wherein a total daily dose of the meloxicam compound is less than or equal to about 7.5 mg.

67. The method of claim 61, wherein the total daily dose of the meloxicam compound is less than about 7.5 mg.

68. The method of claim 61 wherein the pain is acute pain.

69. The method of claim 61, wherein the composition further comprises a second active agent.

70. The method of claim 69, wherein the second active agent includes a member selected from the group consisting of opioids, non-opioid analgesics, antitussives, expectorants, antihistamines, decongestants, 5-HT1 agonists, calcium channel blockers, beta-adrenergic receptor blocking agents, xanthine derivatives, prostaglandin analogs, antacids, proton-pump inhibitors and combinations thereof.

71. The method of claim 69, wherein the composition provides extended release of the second active agent.

72. The method of claim 71, wherein the extended release is sufficient to allow a therapeutically effective dose to be administered at 24 hour intervals.

73. The method of claim 61, wherein the meloxicam compound is meloxicam free acid.

74. The method of claim 61, wherein the meloxicam compound comprises a meloxicam salt with a pharmaceutically acceptable counterion.

75. The method of claim 61, wherein the meloxicam compound comprises a mixture of meloxicam free acid and a meloxicam salt with a pharmaceutically acceptable counterion in which the ratio of meloxicam free acid to total meloxicam ranges from about 0.01 to about 0.99.
